(19) United States
(12) Patent Application Publication
(10) Pub. No.: US 2014/0213985 A1
(43) Pub. Date: Jul. 31, 2014

(54) Securing Means for a Drug Delivery Device

(71) Applicants: Axel Teucher, Frankfurt am Main (DE); Michael Jugl, Frankfurt am Main (DE); German F. Hamm, Frankfurt am Main (DE)

(72) Inventors: Axel Teucher, Frankfurt am Main (DE); Michael Jugl, Frankfurt am Main (DE); German F. Hamm, Frankfurt am Main (DE)

(73) Assignee: Sanofi SAentis Deutschland GmbH, Frankfurt am Main (DE)

(21) Appl. No.: 14/241,774

(22) PCT Filed: Sep. 6, 2012
(86) PCT No.: PCT/EP2012/067389

§ 371 Date: Feb. 27, 2014

(30) Foreign Application Priority Data
Sep. 8, 2011 (EP) 11180535.4

(51) Int. Cl. A61M 5/24; A61M 5/315; A61M 5/31

(52) U.S. Cl. CPC A61M 5/2455; A61M 5/3129; A61M 5/31545
USPC 604/208

(57) ABSTRACT

The present invention relates to a drug delivery device for administering a dose of a medicinal product, comprising: a dose setting and injection mechanism adapted to be operably engaged with a piston of a cartridge containing the medicinal product, at least two housing components being directly and releasably interconnectable to each other, and a flexible coupling means separately coupled to any of said housing components and being adapted to keep the housing components within a predefined spatial range when they are disconnected.

### CROSS REFERENCE TO RELATED APPLICATIONS

The present application is a U.S. National Phase Application pursuant to 35 U.S.C. §371 of International Application No. PCT/EP2012/067389 filed Sep. 6, 2012, which claims priority to European Patent Application No. 11180535.4 filed Sep. 8, 2011. The entire disclosure contents of these applications are herewith incorporated by reference into the present application.

### FIELD OF INVENTION

The present invention relates to the field of drug delivery devices and in particular to injection devices such like pen-type injectors for administering a pre-defined dose of a medicinal product.

### BACKGROUND

Drug delivery devices allowing for multiple dosing of a required dosage of a liquid medicinal product, such as liquid drugs, and further providing administration of the liquid to a patient, are as such well-known in the art. Generally, such devices have substantially the same purpose as that of an ordinary syringe.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

Typically, the medicinal product to be administered is provided in a cartridge that has a moveable piston or bung mechanically interacting with a piston rod of a drive mechanism of the drug delivery device. By applying thrust to the piston in distal direction, a pre-defined amount of the medicinal fluid is expelled from the cartridge.

In particular for elderly or physically infirm users, the overall handling of the device in a home medication environment should be simple and highly reliable. Typically, drug delivery devices and in particular pen-type injectors comprise a multi-component housing. A distal end section which is adapted to be releasably coupled with a needle assembly is typically protected by a protective cap. Moreover, with reusable drug delivery devices, various housing components, such as a pen body and a cartridge holder have to be temporally disconnected in order to replace an empty cartridge.

In practical use, it may happen, that some of these housing components get lost or immobilize in the course of a cartridge replacement procedure. For instance, the pen body housing, the cartridge holder and/or the protective cap due to their overall cylindrical geometry may roll away and may drop down from a support structure like a table. In this way, individual and disassembled components of the drug delivery device may get lost and the end user laboriously has to look for them.

Not only in the course of a cartridge replacement but also for each dose dispensing action, a removable needle assembly is typically to be screwed onto the distal end section of the cartridge holder in order to penetrate a piercable septum of the cartridge that serves as a distal seal. When the needle assembly is removed from the cartridge holder, the septum may become subject to contamination. In order to protect the distal end section of a cartridge holder, a protective cap can be attached thereto in order to protect the cartridge and its septum as soon as the needle holder is disassembled. For the end user, such a protection cap may seem to be superfluous, and it may therefore be handled without due care. Hence, the user may forget or even ignore to put the protective cap onto the distal end of the cartridge holder after a dose dispensing procedure.

Moreover, components of a drug delivery device are particularly susceptible to damage when disassembled, in particular when dropping down in an uncontrolled way.

It is therefore an object of the present invention to provide an improved drug delivery device being less susceptible to damage and which provides easy and unambiguous handling of its various components when disassembled, e.g. for the purpose of a cartridge replacement. It is another object to encourage or even to oblige the user to handle the drug delivery device with due care and to make use of provided protection caps.

### SUMMARY

The drug delivery device according to the present invention is adapted for administering a dose of a medicinal product or medicament. Preferably, the drug delivery device is a pen-type injector and may be releasably coupled with a hypodermic needle in order to administer the product by way of injection. The drug delivery device comprises a dose setting and injection mechanism typically having a piston rod adapted to be operably engaged with a piston of a cartridge that contains the medicinal product.

The cartridge is typically sealed in proximal direction by way of the axially displaceable piston. At its opposite end section, the cartridge is typically sealed by some kind of pierceable sealing structure, like a flexible septum for receiving an injection needle or cannula, thus providing an outlet for the medicinal product when distally directed pressure is exerted on the piston of the cartridge.

The drug delivery device further has at least two housing components that are and releasably interconnectable to each other, wherein a flexible coupling means is separately coupled to any of said housing components. The flexible coupling means is adapted to keep the housing components within a predefined spatial range with respect to each other when the at least two housing components are disconnected. Said at least two housing components are adapted to be interconnected either directly or by way of intermediate housing adapters or other housing components.

The flexible coupling means according to the present invention provides a kind of securing means for the drug delivery device and prevents uncontrolled separation of the device's housing components when disassembled. However, the coupling means still allows for a disengagement and disconnecting of the housing components, e.g. for the purpose of replacing an empty cartridge and/or for removing a protective cap. Since the coupling means is rather flexible, the various housing components can be mutually arranged and displaced almost arbitrarily within a pre-defined spatial range, which is governed by the length, extension or configuration of the flexible coupling means.

The coupling means prevents uncontrolled separation of housing components of the drug delivery device, which in this way can no longer get lost. Furthermore, a risk of damage of particular components of the drug delivery device can be reduced accordingly. In a preferred embodiment, the coupling means comprises a flexible cord or strap which is permanently fastened to any of said housing components at selected fastening points. By way of a flexible cord as a flexible coupling means, a cost-attractive securing means can be provided, which is easy and intuitive in handling and initial assembly.

In a further preferred aspect, a section of the coupling means extending between two adjacent fastening points of different housing components is longer than the distance between the fastening points when the housing components are directly interconnected to each other. In this way, directly interconnectable housing components can be disassembled and separated from each other within a pre-defined range governed by the extension or length of said section of the coupling means. When disassembled each one of the said housing components remains individually connected to the coupling means.

In another aspect, the fastening means is clamped, adhesively fastened, bonded, integrally bonded and/or knotted to at least one of the housing components. The fastening of the cord to the various housing components may further depend on the particular geometry and function of the respective housing component. Where for instance the fastening point is located in an area difficult to access, adhesive fastening, bonding or welding is preferred. Other housing components may feature a lug or an eyelet, allowing to fasten the cord by way of knotting.

Instead of an elastic cord, the coupling means might be integrally formed with at least one of the at least two housing components. When sufficiently flexible, the coupling means may even be integrally formed with both housing components and may keep the housing components within a confined area when disconnected from each other.

In a further and typical aspect, the housing components comprise a cartridge holder adapted to receive the cartridge, a body adapted to house the drive mechanism and/or a protective cap. Typically, body and cartridge holder are directly interconnected to each other. The protective cap is typically adapted to be mounted on the cartridge holder. For this purpose, the cap and cartridge holder may comprise mutually corresponding fastening means, like threads or mutually engaging clip-elements. Alternatively, the protective cap may also be directly connectable to the body, e.g. by way of a snap-fit feature or by way of a threaded engagement.

According to a further aspect, the protective cap is undetachably connected to the cartridge holder by way of the coupling means. In this embodiment, the protective cap can be disconnected from the cartridge holder and can be displaced into an idle position in which a distal end portion of the cartridge holder becomes accessible for a needle holder, which is to be connected thereto.

In still another aspect the at least two housing components are positively, frictionally and/or threadedly engaged. Irrespective of their mutual engagement, the at least two housing components remain permanently interconnected by way of the coupling means.

In a further preferred embodiment, the coupling means is fastened at an inside wall of the protective cap and/or at an inside wall of the body. Here, the coupling means is preferably integrally bonded with said housing components. Since the housing components typically comprise injection molded plastic, fastening of the coupling means to at least one housing component could be conducted in the course of an injection molding process during manufacture of the housing component itself. In this way, a firm and permanent interlock of coupling means and housing components could be attained.

In a further aspect, the drug delivery device and/or one of its housing components comprises a winding mechanism adapted to roll-up the flexible coupling means. Preferably, the winding mechanism is disposed inside a cupped distal end of the protective cap. By way of the winding mechanism, the range, in which the various housing component can be positioned can be enlarged according to the preferences of the respective end-user. Moreover, and on demand, the coupling means can be rolled-up, wherein the overall length of the coupling means is reduced to a range that corresponds to the assembly configuration of the device's housing components.

In its rolled-up configuration, the coupling means also prevents an effective protection against uncontrolled disassembly of the drug delivery device. Disassembling of the device and/or disconnecting of selected housing components then requires to initially release the coupling means, e.g. by releasing or deactivating the winding mechanism.

Retracting of the winding mechanism can be triggered by e.g. temporally applying a tensile force to the flexible coupling means, e.g. to the cord. Subsequent and immediate disengaging of the cord then triggers an autonomous wind-up procedure of the winding mechanism, which may be spring-biased.

In a further preferred embodiment, the drug delivery device comprises at least a first and a second coupling means, each of which pair-wise coupling first and second housing components and/or first and third housing components and/or second and third housing components. Depending on their functionality and with respect to the frequency the housing components are typically disassembled, a mechanical and flexible coupling of pairs of housing components can be individually and selectively modified.

According to a further preferred embodiment, the coupling means is integrally formed with at least one of the housing components. It is even conceivable, that the coupling means is integrally formed with both housing components. This way, first and second housing components together with the interconnecting coupling means may be integrally or unitary formed. Preferably, first and second housing components as well as the coupling means are manufactured by way of injection molding, in particular, when said housing component and/or the coupling means are made of a thermoplastic material.

This way, the housing components are already interconnected when manufactured and separate steps for interconnecting said housing components do not have to be executed.

In still another aspect, the coupling means comprises a film hinge or integral hinge allowing to pivot first and second housing components with respect to each other. In particular, a protective cap integrally formed with a cartridge holder can be disconnected from the distal end of the cartridge holder and may still remain interconnected to the cartridge holder by the film hinge. In this disassembled but connected configuration, a needle assembly or needle holder can be mounted on the distal end section of the cartridge holder in order to conduct a dose setting and dose dispensing procedure.

After dispensing of a dose, and after removal of the needle assembly, the drug delivery device may be stored away in an appropriate storage case providing a support or receptacle precisely adapted and fitting to the geometry of the drug delivery device. The receptacle of the storage case may require to put the protective cap back onto the distal end of the cartridge holder. Otherwise, the drug delivery device may not fit into said storage case support. Since the protective cap is undetachably connected to the cartridge holder, the user is obliged to disassemble the needle assembly and to assemble the protective cap back onto the distal end of the cartridge holder in order to be able to store away the drug delivery device in the provided receptacle of the storage case. Otherwise, if needle assembly and cartridge holder are not disassembled, the protective cap radially protrudes from the cartridge holder and may prevent to store the drug delivery device in the storage case in an appropriate way.

In still another aspect, the invention refers to a drug delivery device, wherein the cartridge is already assembled inside the cartridge holder. In particular, such drug delivery devices can be of disposable type, wherein replacement of the cartridge is not intended. Also with such disposable drug delivery devices, the coupling means may provide a securing means for the pen body housing and the protective cap.

The term “medicament” or “medicinal product”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be apparent to those skilled in the pertinent art, that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, a preferred embodiment of the invention is explained in greater detail by making reference to drawings in which:

FIG. 1 illustrates a first embodiment of a drug delivery device equipped with a coupling means adapted to provide a flexible securing of various housing components of the device,

FIG. 2a shows an alternative embodiment of a cartridge holder provided with a non-detachable protective cap,

FIG. 2b shows a cartridge holder according to FIG. 2a in cross section and

FIG. 2c illustrates the distal end section of the cartridge holder in an enlarged view,

FIG. 3a shows the cartridge holder according to FIGS. 2a to 2c in a perspective illustration with disassembled protection cap,

FIG. 3b shows the embodiment according to FIG. 3a in cross section and

FIG. 3c illustrates the cartridge holder of FIG. 3b in an enlarged view.

### DETAILED DESCRIPTION

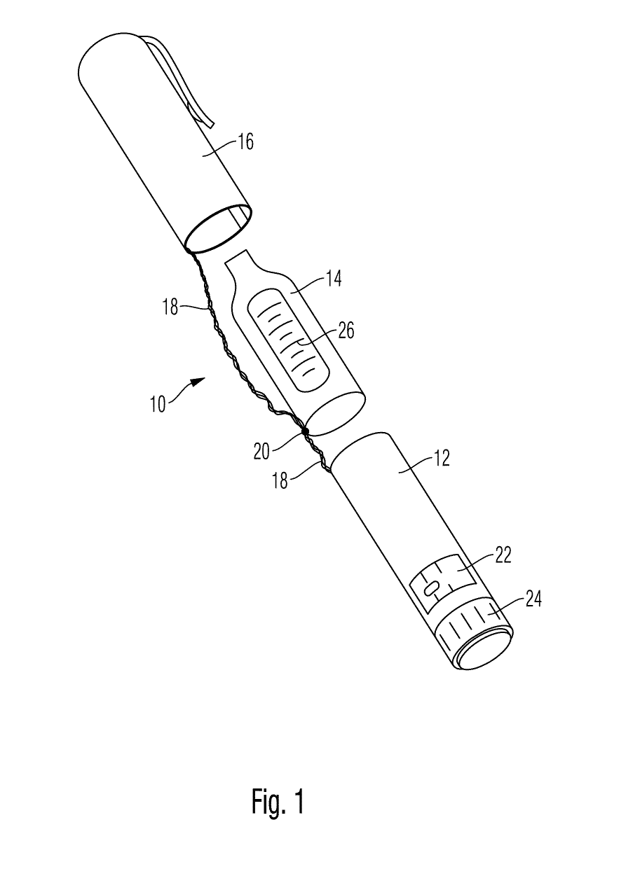

The drug delivery device 10 of pen-injector type is exemplary illustrated in FIG. 1 in a disassembled configuration. The drug delivery device comprises a body 12, a cartridge holder 14 and a protective cap 16. Here, the body 12 comprises a display means 22 as well as a dose selecting and dose dispensing button 24 at its proximal end section. The body 12 is adapted to house a not further illustrated drive mechanism of the drug delivery device 10, which is adapted to exert pressure on a piston slidably arranged in a sealed cartridge containing the medicinal product to be dispensed.

The cartridge, which is not explicitly illustrated in FIG. 1, typically comprises a transparent cavity, allowing to visually control the position of the piston. Additionally, the cartridge holder 14 has an inspection window 26, optionally furnished with a scale allowing to visually determine the filling level of the cartridge.

The distal neck-like portion of the cartridge holder 14, which faces away from the body 12 is adapted to be releasably engaged with a disposable injection needle or with a comparable hypodermic injection means.

The protective cap 16 is to be put over the cartridge holder 14 if the device 10 is not in use. The protective cap may either be connected with the cartridge holder 14 and/or with the body 12 of the drug delivery device. Cartridge holder 14 is typically releasably connected to the distal end portion of the body 12. Mutual and direct interconnection of the illustrated housing components 12, 14, 16 either comprises a threaded engagement or some kind of positive interlock, like a snap-fitted or clipped interconnect.

The flexible coupling means 18 comprises a flexible cord or strap in the embodiment according to FIG. 1. Here, the coupling means 18 is fastened to the cartridge holder 14 at a fastening point 20 in the vicinity of a proximal end section of the cartridge holder 14. However, there are various other positions and options where and how to fasten the coupling means 18 to the cartridge holder 14 or to residual housing components 12, 16.

For the purpose of fastening the coupling means 18 to the cartridge holder, the cartridge holder 14 may comprise an eyelet at the fastening point 20 allowing to knot or to clamp the flexible cord 18 with the cartridge holder 14. Alternatively, fastening of the cord 18 to the cartridge holder 14, to the body 12 and/or to the cap 16 may involve usage of adhesives, application of heat, e.g. by ultrasonic or laser welding or any other suitable interconnection method.

As can be seen in the sketch of FIG. 1, elongation of the flexible coupling means 18 is longer than the axial distance between consecutive or neighbouring fastening points of different housing components 12, 14, 16. In this way, the disassembled and disconnected housing components 12, 14, 16 can be freely and arbitrarily positioned and arranged within a certain and pre-defined spatial range, which allows for instance to replace an empty cartridge and/or to remove the protective cap 16, e.g. for the purpose of an administering procedure.

Even though not explicitly illustrated, the cap 16 at is uppermost and distal end section may comprise a winding mechanism for the cord 18. By way of this mechanism, the spatial range, within which the various housing components 12, 14, 16 can be arranged and positioned, can be varied and enlarged according to a user's preference. In the illustration according to FIG. 1, the housing components 16, 14, 12 are interconnected and coupled to each other by way of a single coupling means 18, such like a cord. Instead and alternative to this embodiment it is even conceivable, that pairs of housing components 12, 14, 16 are separately coupled to each other by way of several coupling means. For instance, the protective cap 16 and the cartridge holder 14 may be coupled by means of a first coupling means and the cartridge holder 14 and the body 12 may be separately coupled by way of another second coupling means. Since removal of the cap is more frequent than disassembling of body 12 and cartridge 14, the respective first and second coupling means may provide different functionality, such like a different mechanical flexibility.

Furthermore, it is conceivable, that a first coupling means connects body 12 and cartridge 14 while a second coupling means individually couples body 12 and cap 16.

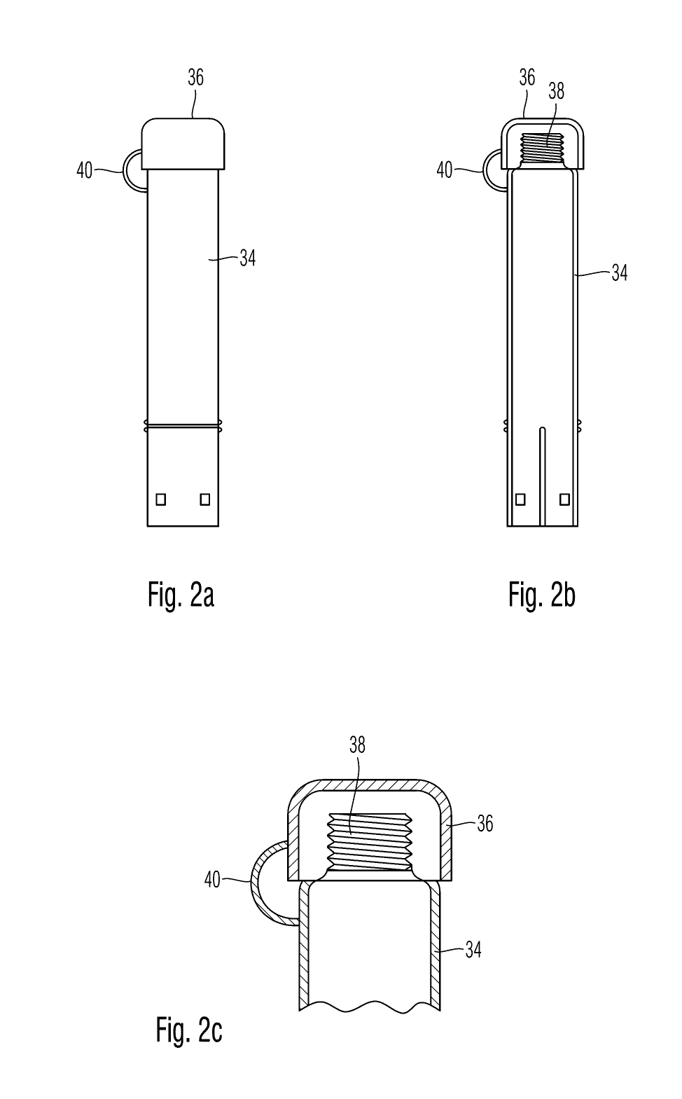

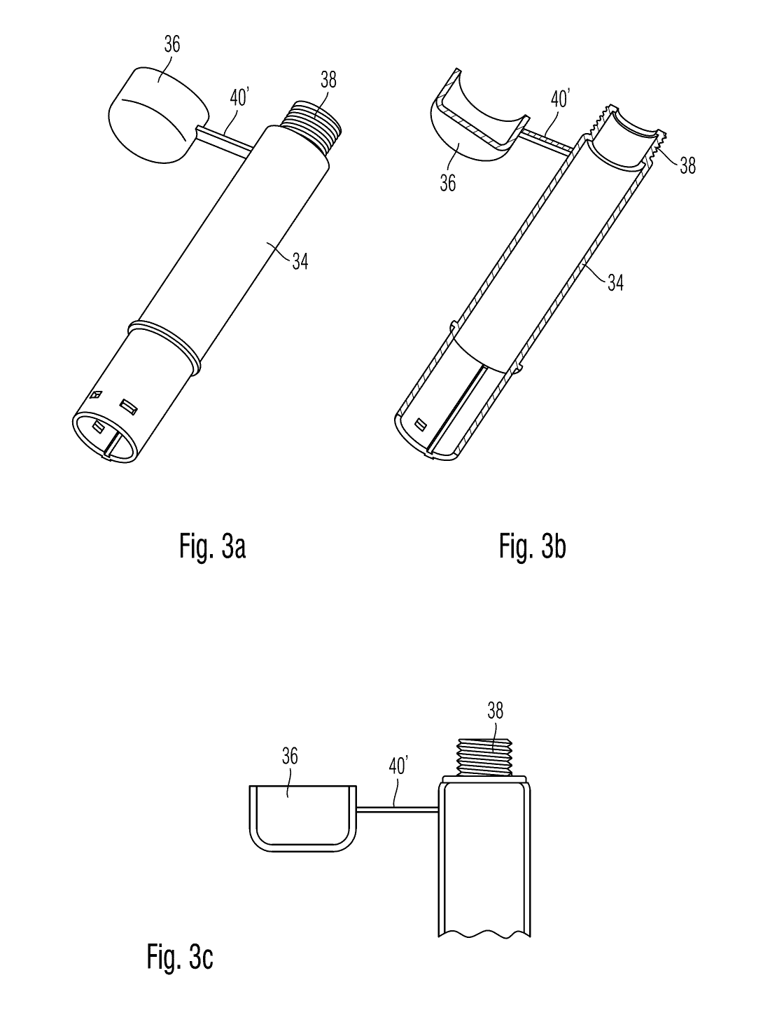

FIGS. 2a, 2b and 2c illustrate another embodiment, wherein a distal end section of a cartridge holder 34 is provided with a non-detachable protective cap 36 being integrally formed with a cartridge holder 34 by way of a film hinge 40. As illustrated in FIGS. 3a, 3b and 3c, the distal end section of the cartridge holder 34 comprises a stepped down and threaded neck portion 38 which is adapted to receive a correspondingly threaded needle or piercing assembly, e.g. having a double tipped injection needle.

The injection needle which is not particularly illustrated here is intended to pierce and to penetrate a septum of a cartridge disposed inside the hollow cylindrical housing of the cartridge holder 34. With its opposite distal tip, the needle is intended to penetrate the skin of a patient for medicament delivery.

The protective cap 36 may be positively and/or frictionally engaged with the distally located socket portion 38 of the cartridge holder 34. Preferably, the protective cap 36 is clipped or snap fitted onto the cartridge holder 34 as illustrated in FIGS. 2a, 2b and 2c This way, the protective cap 36 can be easily transferred into a release configuration as illustrated in FIGS. 3a, 3b and 3c in which the threaded neck portion 38 becomes accessible in order to interconnect a needle assembly and the needle holder 34. The film hinge 40′ in this configuration almost radially extends from the cylindrical cartridge holder 34.

During setting and dispensing of a dose, the protective cap 36 remains attached and fastened to the cartridge holder 34. If for instance the drug delivery device should be stored away in a storage case that provides a receptacle geometrically fitting with the drug delivery device, it may become necessary to put the cap 36 back onto the threaded neck portion 38 so that the cartridge holder or the drug delivery device properly fits into the provided receptacle. This way, the flexible coupling means 40 encourage and oblige the user to return the protective cap 36 into its initial configuration as depicted in FIGS. 2a, 2b and 2c prior to a proper storage of the device in an appropriate case.

Moreover, the non-detachable protective cap 36 may also encourage or even oblige the user to remove a piercing or needle assembly after usage of the device since a pen cap 16 as schematically illustrated in FIG. 1 cannot be put back onto the cartridge holder as long as the protective cap 36 has not returned to its initial and protecting configuration as illustrated in FIG. 2a, 2b or 2c. This way, the end user is required to properly disconnect needle assembly and cartridge holder 34 and to discard the used needle as recommended by the device instructions.

By way of the protecting cap 36 being non-detachably connected to the cartridge holder 34, a regular removal of a used needle assembly and an immediate returning of the protective cap 36 onto the distal end section 38 of the cartridge holder 34 is supported and encouraged.

1-12. (canceled)

13. A drug delivery device for administering a dose of a medicinal product, comprising:
a dose setting and injection mechanism adapted to be operably engaged with a piston of a cartridge containing the medicinal product,
at least two housing components being directly and releasably interconnectable to each other, and characterized by
a flexible coupling means separately coupled to any of said housing components and being adapted to keep the housing components within a predefined spatial range when they are disconnected, wherein the coupling means comprises a flexible cord permanently fastened to at least two of said housing components at selected fastening points.

14. The drug delivery device according to claim 13, wherein a section of the coupling means extending between two adjacent fastening points of different housing components is longer than the distance between the fastening points when the housing components are interconnected.

15. The drug delivery device according to claim 13, wherein the coupling means is clamped, adhesively fastened, bonded and/or knotted to the housing components.

16. The drug delivery device, wherein the coupling means is integrally bonded to at least one of the housing components.

17. The drug delivery device according to claim 13, wherein the housing components comprise a cartridge holder adapted to receive the cartridge, a body adapted to house the drive mechanism and/or at least one protective cap.

18. The drug delivery device according to claim 17, wherein the protective cap is undetachably connected to the cartridge holder by way of the coupling means.

19. The drug delivery device according to claim 13, wherein the at least two housing components are positively and/or threadedly engaged.

20. The drug delivery device according to claim 13, wherein the coupling means is fastened at an inside wall of the protective cap and/or of the body.

21. The drug delivery device according to claim 13, further comprising a winding mechanism adapted to roll-up the flexible coupling means.

22. The drug delivery device according to claim 13, wherein the winding mechanism is disposed inside a cupped distal end of the protective cap.

23. The drug delivery device according to claim 13, comprising at least a first and a second coupling means, each of which pair-wise coupling first and second housing components and/or first and third housing components and/or second and third housing components.

24. The drug delivery device according to claim 13, further comprising a cartridge filled with the medicinal product.
